(19) United States
(12) Patent Application Publication
(10) Pub. No.: US 2021/0177778 A1
(43) Pub. Date: Jun. 17, 2021

(54) EXOSOME PRODUCTION PROMOTER

(71) Applicants: DAICEL CORPORATION, Osaka-shi, Osaka (JP); NATIONAL UNIVERSITY CORPORATION HOKKAIDO UNIVERSITY, Sapporo-shi, Hokkaido (JP)

(72) Inventors: Katsuyuki MUKAI, Tokyo (JP); Kohei YUYAMA, Sapporo-shi (JP); Yasuyuki IGARASHI, Tokyo (JP)

(73) Assignees: DAICEL CORPORATION, Osaka-shi, Osaka (JP); NATIONAL UNIVERSITY CORPORATION HOKKAIDO UNIVERSITY, Sapporo-shi, Hokkaido (JP)

(21) Appl. No.: 16/613,906

(22) PCT Filed: May 16, 2018
(86) PCT No.: PCT/JP2018/018981

§ 371 Date: Nov. 15, 2019

(30) Foreign Application Priority Data
May 16, 2017 (JP) 2017-097551

(51) Int. Cl. A61K 31/164; A23L 33/115; A61P 25/28

(52) U.S. Cl. CPC A61K 31/164; A61P 25/28; A23L 33/115

(57) ABSTRACT

The purpose of the present invention is to provide an agent for promoting exosome production with which exosome production can be easily promoted.

An agent for promoting exosome production using ceramide as an active ingredient

### TECHNICAL FIELD

The present invention relates to a drug capable of promoting exosome production.

### BACKGROUND ART

At present, Alzheimer's disease is a neurodegenerative disease that accounts for more than half of dementia causes, and it has been desired to establish methods for treating and preventing the Alzheimer's disease. A pathologic mechanism of the Alzheimer's disease has been elucidated through studies so far, and in order to prevent the Alzheimer's disease, suppress the progression of the Alzheimer's disease, or improve the Alzheimer's disease, it is thought that one of the important points is to inhibit an accumulation of amyloid β protein which is the center of the pathological mechanism.

Drugs that suppress the accumulation of the amyloid β protein have been extensively researched so far. For example, Patent Document 1 discloses an inhibitor that suppresses a formation of amyloid fibril containing a water-soluble peptide metal complex array. In addition, for example, Patent Document 2 discloses a composition for inhibiting aggregation of amyloid β protein which uses a perilla extract as an active ingredient. Further, for example, Patent Document 3 discloses an amyloid β aggregation inhibitor caused by a GNE gene mutation which contains at least one of N-acetylneuraminic acid, N-acetylmannosamine, and sialyl lactose.

However, recent studies have suggested that exosomes are involved in the metabolism of amyloid β protein in a brain and the expression and progression of the Alzheimer's disease. The exosomes are membrane endoplasmic reticulum having a diameter of 30 to 200 nm surrounded by lipid bilayers secreted from cells, contain nucleic acids such as mRNA and miRNA, proteins, or the like, and serve as a bearer of intercellular communication.

It is thought that the amyloid β protein migrates to microglia via the exosomes and then is decomposed. For example, Non-Patent Document 1 suggests that nerve cell-derived exosomes have the ability to highly express amyloid β-binding glycolipid and capture amyloid β protein and the action to remove the amyloid β protein in cooperation with phagocyte microglia in a brain. In addition, Non-patent document 2 suggests that neuron-derived exosomes capture amyloid β on a surface thereof and transport the captured amyloid β to microglia to increase the decomposition efficiency of amyloid β protein. In addition, for example, Non-Patent Document 3 suggests that the secretion of the exosomes promotes the removal function of the amyloid β protein by the microglia. In addition, for example, Non-Patent Document 4 suggests that exosomes are involved in the metabolism of amyloid β in a brain.

It has been known that the exosomes are involved in the removal and accumulation suppression of the amyloid β protein. Therefore, it is thought that promoting the production of the exosomes can suppress the accumulation of the amyloid β protein, and is effective to prevent and treat the Alzheimer's disease.

However, drugs that can promote the production of the exosome have not yet been studied sufficiently. Moreover, it has not been known so far that ceramide can promote the production of the exosomes.

### PRIOR ART DOCUMENTS

### Patent Documents

Patent Document 1 Japanese Patent Laid-Open Publication No. 2016-145175

Patent Document 2 Japanese Patent Laid-Open Publication No. 2016-124865

Patent Document 3 Japanese Patent Laid-Open Publication No. 2014-224132

### Non-Patent Documents

Non-Patent Document 1: Monthly “Cell”, published by New Science, 2016, 48 (1), pp. 44-47
Non-Patent Document 2: Dementia Japan 30, Vol. 30, No. 3, 358-367 (2016)
Non-Patent Document 3: Yuyama K et al., J Biol Chem., 289 (35), 24488-98 (2014)
Non-Patent Document 4: Yuyama K et al., J Biol Chem., 287 (14), 10977-89 (2012)

### SUMMARY OF THE INVENTION

### Problems to be Solved by the Invention

The present invention has been made in view of the above situations, and an object of the present invention is to provide an agent for promoting exosome production capable of easily promoting a production of an exosome in vivo.

### Means for Solving the Problem

The present inventor has conducted intensive studies to solve the above problems, and found that ceramide can promote the production of the exosomes. In addition, it has been found that the production of the exosomes can be more effectively promoted particularly when fatty acid constituting ceramide has 6 to 26 carbon atoms. The present invention has been completed by further investigation based on these findings.

That is, the present invention provides the inventions of aspects listed below.

Item 1-1. An agent for promoting exosome production comprising a ceramide as an active ingredient.

Item 1-2. The agent for promoting exosome production according to Item 1-1, wherein fatty acid constituting the ceramide has 6 to 26 carbon atoms.

Item 1-3. The agent for promoting exosome production according to Item 1-1 or 1-2, wherein a sphingoid moiety constituting the ceramide has 18 carbon atoms.

Item 1-4. The agent for promoting exosome production according to any one of Items 1-1 to 1-3, wherein the agent for promoting exosome production is a food and drink for promoting a production of an exosome.

Item 1-5. The agent for promoting exosome production according to any one of Items 1-1 to 1-3, wherein the agent for promoting exosome production is a pharmaceutical product for promoting a production of an exosome.

Item 1-6. The agent for promoting exosome production according to any one of Items 1-1 to 1-3, wherein the agent for promoting exosome production is an agent for preventing Alzheimer's disease.

Item 2-1: Use of ceramide for a production of an agent for promoting exosome production.

Item 2-2. Use according to Item 2-1, wherein fatty acid constituting the ceramide has 6 to 26 carbon atoms.

Item 2-3. Use according to Item 2-1 or 2-1, wherein a sphingoid moiety constituting the ceramide has 18 carbon atoms.

Item 2-4. Use according to any one of Items 2-1 to 2-3, wherein the agent for promoting exosome production is a food and drink for promoting a production of an exosome.

Item 2-5. Use according to any one of Items 2-1 to 2-3, wherein the agent for promoting exosome production is a pharmaceutical product for promoting a production of an exosome.

Item 2-6. Use according to any one of Items 2-1 to 2-3, wherein the agent for promoting exosome production is an agent for preventing Alzheimer's disease.

Item 3-1. A method for promoting a production of exosomes, comprising administering or ingesting an effective amount of ceramide to a person who needs to promote the production of the exosome.

Item 3-2. The method for promoting a production of an exosome according to Item 3-1, wherein fatty acid constituting the ceramide has 6 to 26 carbon atoms.

Item 3-3. The method for promoting a production of an exosome according to Item 3-1 or 3-2, wherein a sphingoid moiety constituting the ceramide has 18 carbon atoms.

Item 3-4. The method for promoting a production of an exosome according to any one of Items 3-1 to 3-3, wherein a food or drink containing the ceramide is ingested.

Item 3-5. The method for promoting a production of an exosome according to any one of Items 3-1 to 3-3, wherein a pharmaceutical product containing the ceramide is administrated.

Item 3-6. The method for promoting a production of an exosome according to any one of Items 3-1 to 3-3, wherein the person who needs to promote the production of the exosome is a person who needs to prevent Alzheimer's disease, and is administrated with or ingests the effective amount of the ceramide for prevention of the Alzheimer's disease.

Item 3-7. The method for promoting a production of an exosome according to Item 3-6, wherein the person who needs to prevent the Alzheimer's disease is a person who is concerned about an onset of familial Alzheimer's disease.

### Advantages of the Invention

According to the present invention, it is possible to provide the agent for promoting exosome production capable of easily promoting the production of the exosome in vivo. Further, the agent for promoting exosome production of the present invention can be administered or ingested in the form of the oral administration and the food and drink and therefore is non-invasive, and therefore the agent for promoting exosome production is less burden on the patients and can easily promote the production of the exosome in vivo.

### BRIEF DESCRIPTION OF THE DRAWINGS

FIG. 1 is a graph showing a number of exosome particles after 24 hours when ceramide (Cer), sphingomyelin (SM), or glucosylceramide (GluCer) is added.

FIG. 2 is a graph showing a number of exosome particles after 24 hours when konjac ceramide (kCer) or konjac glucosylceramide (kGluCer) is added at 5 μM, 7.5 μM, and 10 μM.

FIG. 3 is a graph showing results obtained by measuring a concentration of amyloid β protein (Aβ1-40) in a culture supernatant after the exosome produced under the presence of konjac ceramide (kCer) and konjac glucosylceramide (kGluCer) or under the absence of sphingolipid and the amyloid β protein (Aβ1-40) are added to mouse microglia cells and cultured for 24 hours.

### EMBODIMENTS OF THE INVENTION

The present invention relates to an agent for promoting exosome production using ceramide as an active ingredient. Hereinafter, the agent for promoting exosome production of the present invention will be described in detail.

Ceramide

An agent for promoting exosome production of the present invention contains a ceramide as an active ingredient.

The ceramide is a compound having a structure in which fatty acid is amide-bonded to sphingoid. In the ceramide used in the present invention, a structure of a sphingoid moiety is not particularly limited, but the sphingoid moiety constituting the ceramide may have 18 carbon atoms.

In the ceramide used in the present invention, specific examples of the structure of the sphingoid moiety may include 4-sphingenin (sphingosine), 4-hydroxysphinganin (phytosphingosine), 4-hydroxy-trans-8-sphingenin, 4-hydroxy-cis-8-sphingenin, sphinganine, trans-8-sphingenin, cis-8-sphingenin, trans-4-sphingenin, trans-4,trans-8-sphingadienine, trans-4,cis-8-sphingadienine, cis 4-cis 8-sphingadienine, cis 4-trans 8-sphingadienine, and the like. Among these, preferably, trans-4,cis-8-sphingadienine, cis 4-cis 8-sphingadienine, trans 4-trans 8-sphingadienine, cis 4-trans 8-sphingadienine, 4-hydroxy-cis-8-sphingenin, and 4-hydroxy-trans 8-sphingenin.

In the ceramide used in the present invention, the carbon atoms of the fatty acid bonded to the sphingoid moiety are not particularly limited, but may be 6 to 26, preferably 6 to 24, and more preferably 6 to 18. In addition, the fatty acid may be any of a saturated fatty acid, an unsaturated fatty acid containing a carbon-carbon double bond and/or a carbon-carbon triple bond, and an α-hydroxy fatty acid.

In the ceramide used in the present invention, specific examples of the fatty acid bonded to the sphingoid moiety include hexanoic acid (C6:0), octanoic acid (C8:0), decanoic acid (C10:0), dodecane acid (C12:0), tetradecanoic acid (C14:0), hexadecanoic acid (C16:0), octadecanoic acid (C18:0), icosanoic acid (C20:0), heneicosanoic acid (C21:0), docosanoic acid (C22:0), tricosanoic acid (C23:0), tetradocosanoic acid (C24:0), pentacosanoic acid (C25:0), hexadocosanoic acid (C26:0), heptacosanoic acid (C27:0), octadocosanoic acid (28:0), cis-9-octadecenoic acid (C18:1), and the like. Note that in the notation “CX:Y” shown in parentheses of the fatty acid, CX represents carbon atoms per molecule, Y represents the number of unsaturated bonds per molecule, for example, “C16:0” represents fatty acid in which the carbon atoms are 16 and the number of unsaturated bonds is 0. Among these, examples of the fatty acids preferably include octadecanoic acid, docosanoic acid, and icosanoic acid.

The ceramide may be derived from either a plant or an animal, but is preferably derived from a plant since the plant is higher in safety.

Specific examples of the derived plants of the ceramide used in the present invention include almonds, sea lettuces, green laver, fat hen, acacia, madder, red grape, Japanese red pine (including pine resin, amber, and copal. The same applies to pines below), almond mushroom, indian lettuce, chocolate vine, japanese morning glory, azalea, hydrangea, Angelica, azuki bean, asparagus, acerola, gambir, anise, avocado, amakusa, amacha, five-leaf ginseng, amanatsu, amaryllis, althaea, amica, aloe, angelica, apricot, angkor, benzoin, rush, izayoi rose, japanese yew, fig tree, ginkgo, iyokan, ylang ylang, fennel, oolong tea, turmeric, common mallow, prunella, oudo, japanese apricot, Quercus salicina, citrus unshiu, rose fruit, shallots, Eleutherococcus senticosus, cytisus, enokitake, elderflower, pea, orchid, ougonkan, chinese plantain, great white thistle, barley, Atractylodes japonica, osmanthus, Hypericum erectum, Lamium album, Dioscorea tokoro, olive, oregano, orange (including orange peel), carnation, cacao, persimmon, alehoof, cocktail fruit, pueraria root, daimyo oak, katakuri, pumpkin, chamomile, camu camu, chamomile, Trichosanthes cucumeroides, japanese larch, cara mandarin, chinese quince, garcinia, cardamom, kawachi bankan, campei, bramble, kiwi, balloon flower, cabbage (including kale), caraway, cucumber, kiyomi, kumquat, ginkgo, guava, chinese desert-thorn, kudzu, common gardenia, cumin, cranberry, walnut, grapefruit, clementine, clove, black pine, black bean, chlorella, cassia seed, geranium herb, cowberry, pepper, cosmos, burdock, wheat (including wheat germ), sesame, komatsuna, rice (including rice bran), coriander, konjac (konjac potato) (including konjac powder), kombu, salmon berry, cypress, pomegranate, sweet potato, eddoe, sugarcane, sugar beet, saffron crocus, pomelo, haws, Japanese pepper, shiitake, cyclamen, red shiso, beech mushroom, potato, Chinese peony, jasmine, job's tears, crown daisy, ginger, sweet flag, bamboo-leaf oak, winter daphne, cinnamon, watermelon, sweet pea, sweet spring, field horsetail, star anise, cainito, sudachi, sweetleaf, Japanese plum, common sage (scarlet sage), setoka, common mallow, seminole, celery, cinidum rhizome, swertia herb, buckwheat, broad bean, Japanese radish, soybean (including soybean curd refuse), bitter orange, thyme, bamboo shoot, onion, golden oyster mushroom, tarragon, taro, tankan, tangor, Chinese red sage, tangelo, dandelion, chicory, oenothera tetraptera, horsetail, camellia, indian pennywort, clover, climbing plant, New Zealand spinach, leopard plant, dill, dekopon, pelargonium (geranium), benincasa cerifera, chile pepper, Japanese angelica root, caterpillar fungus, corn, houttuynia, ipecacuanha, eucommia bark, Japanese ash, Chinese yam, shepherd's purse, citrus unshiu, Japanese summer orange, nutmeg, heavenly bamboo, bitter melon, worm wood, oriental garlic, carrot, garlic, welsh onion, Chinese yarrow, saw palmetto, wild rocambole, vervain, palm, pineapple, hibiscus, Stellaria media, basil, parsley, naked barley, citrus hassaku, mint, adlay, banana, giant crape-myrtle, vanilla, paprika, hamamelis, haruka, harumi, hallehime, citrus grandis, beet, pimento, red spider lily, trapa japonica, hizikia, pistachio, hyssop (hyssopus officinalis), daisy, corn poppy, hinoki cypress, cypress, castor, sunflower, himenotsuki, citrus tamurana, Japanese loquat, orchid, sicklefruit fenugreek, fuki, blackberry, plum, blueberry (including billberry), prune, pomelo, luffa, safflower, benimadonna, deadly nightshade, bergamot, rose balsam, spinach, Chinese lantern plant, ficus religiosa, peony, hop, jojoba, ponkan, hen-of-the woods, ephedra, maca, macadamia nut, murcott tangor, silvervine, marigold, marihime, mango, Japanese honeywort, tangelo, mimosa, myoga, myrrh, murasaki, mace, lemon balm, melilot, melon, men (including cotton seed oil cake), etiolated seedling, rodger's bronze leaf, Japanese yam, gold-banded lily, sage, eucalyptus, strawberry geranium, yuzu, lily, coix seed, yomena (aster), mugwort, lime, rye, lilac, raspberry, peanut, rakkyo, apple (including apple fiber), Japanese gentian, reiko, lychee, lettuce, lemon, Chinese milk vetch, lotus root, rose hip, rosemary, bay leaf, tree onion, wasabi (including horseadish), and the like Among these, examples of the derived plants of the ceramide preferably include wheat, rice, corn, soybean, konjac, hen-of-the woods, golden oyster mushroom, citrus fruits (for example, citrus unshiu, amanatsu, iyokan, orange, cara mandarin, kiyomi, Japanese summer orange, tangor, citrus hassaku, citrus tamurana, pomelo, and the like), and more preferably konjac.

Specific examples of the derived animals of the ceramide used in the present invention include all or part of tissues of echinoderms and molluscs such as sea urchins, starfish, octopus, and squid, brain tissues and skin tissues of mammals such as horse and cow, milk from mammals such as human, cow, and goat and processed products such as fermented products thereof, and the like.

The ceramide can be obtained from the above-mentioned derived plants or animals by the publicly-known extraction methods. In addition, the ceramide may be obtained as an enzyme-treated product of glycosphingolipid. In addition, the ceramide is commercially available, and commercially available products may be used.

Examples of the enzyme-treated product of the glycosphingolipid include extracts of the derived plants described above, concentrates thereof, enzyme-treated products of purified products obtained by purifying the concentrates, or the like.

The glycosphingolipid is glycolipid in which sugar is bonded to a primary alcoholic hydroxy group of the ceramide such as glucosylceramide or lactosylceramide. The glycosphingolipid is not particularly limited as long as the ceramide described above can be obtained, and any glycosyl such as glucose, galactose, or sugar chain may be bonded to the ceramide. The glycosphingolipid can be obtained from the above-mentioned derived plants by the publicly-known extraction methods. In addition, the glycosphingolipid is commercially available, and commercially available products may be used.

The enzyme used for the enzyme treatment of the glycosphingolipid is not particularly limited as long as it is an enzyme that hydrolyzes the bond between the sugar chain and ceramide of the glycosphingolipid, and examples thereof include endoglycoceramidase (EGCase).

As the EGCase, three molecular species (EGCase I, EGCase II, and EGCase III) having different isoelectric points and molecular weights are known, and it has been known that substrate specificity differs depending on the molecular species. The molecular species of the EGCase to be used may be appropriately set according to the structure of the glycosphingolipid as the substrate. For example, as the glycosphingolipid, the EGCase I is suitably used in the case of cerebroside, particularly konjac-derived glycosphingolipid. The conditions for the enzyme treatment may be appropriately selected so that a desired enzyme reaction is performed.

Examples of the concentration method of the extract include the publicly-known concentration method using a vacuum concentration device such as an evaporator. In addition, examples of the purification method include publicly-known purification methods such as alkali treatment, solvent fractionation, and silica gel chromatography.

After the enzyme treatment, the enzyme-treated product may be used as it is, those obtained by drying a residue obtained by solid-liquid separation of the enzyme-treated product, those obtained by drying the solid-liquid separated residue, those obtained by drying a reaction product as it is, or the like may be used. In addition, by separating the enzyme-treated product into solid and liquid, adding water to the product, and then separating the product into solid and liquid again, the enzyme-treated product may be cleaned to remove impurities.

In the agent for promoting exosome production of the present invention, as the ceramide, those having one structure or origin may be used alone, or those having two or more structures or origins may be used in combination.

In the agent for promoting exosome production of the present invention, the “using the ceramide as the active ingredient” means that the ceramide acts on target cells that promote the production of the exosome, and the ceramide itself is not only used as an active ingredient, but components that promote the production of the exosome by being ingested or administrated and subjected to metabolism, decomposition, resynthesis, and the like to generate ceramide in vivo can also be used as an active ingredient. For example, most ceramides derived from plants exist in a glucosylated form, but if the glucosylated ceramide is ingested orally, the glucosylated ceramide is subjected to metabolism, decomposition, and resynthesis to produce ceramide in vivo, and the ceramide can act on the target cell, and as a result, can be used the active ingredient in the agent for promoting exosome production of the present invention. Specifically, when the glucosylated ceramide is ingested orally, glucose is liberated by hydrolysis in a small intestine, and may be further decomposed into sphingoid and fatty acid, but the sphingoid and the fatty acid are bonded until getting into the blood and resynthesized with ceramide, so the ceramide can promote the production of the exosome to the target cell.

A content of the ceramide in the agent for promoting exosome production of the present invention is not particularly limited as long as the content is an effective amount that can promote the production of the exosome in vivo, and is appropriately adjusted according to a usage, a dosage form, an administration form, and the like.

Other Additive Components

The agent for promoting exosome production of the present invention may contain other additive components according to the dosage form in the range in which the effects of the present invention are not impaired, in addition to the above-mentioned ceramide. Examples of the additive components that can be contained in the agent for promoting exosome production of the present invention include water, fats and oils, waxes, hydrocarbons, fatty acids, higher alcohols, esters, plant extracts, water-soluble polymers, surfactants, metal soaps, alcohols, polyhydric alcohols, pH adjusters, antioxidants, ultraviolet absorbers, preservatives, fragrances, powders, thickeners, dyes, chelating agents, and the like. These additive components may be used alone or in combination of two or more. In addition, the content of the additive components is appropriately set according to the type of additive components to be used, the dosage form of the agent for promoting exosome production of the present invention, and the like.

Dosage Form/Formulation Form/Usage

The dosage form of the agent for promoting exosome production of the present invention is not particularly limited, and may be any of a solid shape, a semi-solid shape, or a liquid shape, and appropriately set according to the type, the usage, the administration method, and the like of the agent for promoting exosome production.

The administration method of the agent for promoting exosome production of the present invention is not particularly limited, and may be appropriately selected depending on the type of diseases to be applied, and may be administrated for the entire body or locally administrated. Specific examples of the administration method include oral, intravascular (intraarterial or intravenous), transdermal, enteral, pulmonary, intranasal administrations, and the like. The intravascular administration also includes intravascular injection and continuous intravenous infusion. Of these, the oral administration, the intravascular administration, and the intranasal administration are preferred from the viewpoints of easy administration and effective promotion of the production of the exosome.

The formulation form of the agent for promoting exosome production of the present invention is not particularly limited, and can be appropriately set to the formulation form suitable for the administration method, and examples of the formulation form include any formulation forms such as tablets, capsules, granules, powders, syrups, injections, instillation, and suppositories. For example, when the administration form of the agent for promoting exosome production of the present invention is administrated orally, the administration form is not particularly limited as long as the oral administration is possible, and specific examples thereof include foods and drinks and medicines for internal use.

When the agent for promoting exosome production of the present invention is used in the formulation form of the food or drink products, the agent for promoting exosome production of the present invention may be prepared in a desired form as it is or in combination with other food materials or additive components. Examples of such foods and drinks include foods for specified health use, nutritional supplementary foods, functional foods, foods for patients, and the like in addition to general foods and drinks. The form of these foods and drinks is not particularly limited, but specific examples thereof include supplements such as capsules (soft capsules, hard capsules), tablets, granules, powders, jellies, and liposome preparations; drinks such as energy drink, fruit juice drink, carbonated drink, and lactic acid drink; favorite items such as dumplings, ice cream, sherbet, gummy, and candy, and the like. Among these foods and drinks, the drinks and the supplements are preferable, and the drinks and the capsules are more preferable.

When the agent for promoting exosome production of the present invention is used in the formulation form of the medicines for internal use, the agent for promoting exosome production of the present invention may be prepared in a desired form as it is or in combination with other additive components. Specific examples of the medicines for internal use include drinks, capsules (soft capsules, hard capsules), tablets, granules, powders, jellies, syrups, liposome formulations, and the like. Among these medicines for internal use, the capsules and the drinks are preferable.

When the agent for promoting exosome production of the present invention is in the formulation form of the food or drink or the medicines for internal use, the content of the ceramide that is the active ingredient is not particularly limited as long as the content is an effective amount that promotes the production of the exosome, and the content may be appropriately set depending on the formulation form, but examples thereof include 1 to 20% by mass or the like, preferably 3 to 10% by mass, and more preferably 6 to 8% by mass.

The agent for promoting exosome production of the present invention can be applied to diseases whose symptoms are reduced or improved based on the promotion of the production of the exosome. For example, since the exosome can suppress the accumulation of the amyloid β protein which is one of the causes of the onset of the Alzheimer's disease, if the production of the exosome is promoted, it is thought that the onset of the Alzheimer's disease can be suppressed or the symptoms can be reduced. That is, the agent for promoting exosome production of the present invention can suppress the onset of the Alzheimer's disease or reduce the symptoms. Thus, the agent for promoting exosome production of the present invention can also be suitably applied as an Alzheimer's disease preventive agent. In particular, those who are relatives who have familial Alzheimer's disease but do not have familial Alzheimer's disease are routinely required to prevent the familial Alzheimer's disease, so that the agent for promoting exosome production of the present invention is suitably applied to those who are concerned about the onset of such familial Alzheimer's disease.

In addition, examples of other diseases whose symptoms can be improved by promoting the production of the exosome include neurodegenerative diseases such as Parkinson's disease, frontotemporal degeneration, and polyglutamine disease.

The application amount of the agent for promoting exosome production of the present invention is not particularly limited, and an effective amount for promoting the production of the exosome may be appropriately set according to the formulation form, the usage, the administration target, the expected effect, and the like. For example, when the agent for promoting exosome production of the present invention is orally ingested or administered, examples of the ingestion or dosage amount include 0.6 to 10 mg and preferably 0.6 to 1.2 mg per day for an adult in terms of ceramide. The agent for promoting exosome production of the present invention may be ingested or administered once or several times so that the amount per day is in the above-mentioned range.

As described above, the agent for promoting exosome production of the present invention containing the ceramide can promote the production of the exosome. The agent for promoting exosome production of the present invention can be used for the treatment or prevention of diseases whose symptoms are improved by promoting the production of the exosome. Specifically, for example, the agent for promoting exosome production of the present invention can be used for prevention or treatment of neurodegenerative diseases and the like such as Alzheimer's disease (including familial Alzheimer's disease), Parkinson's disease, frontotemporal degeneration, and polyglutamine disease.

### EXAMPLES

Next, the present invention will be described in detail with reference to Examples, but is not limited to these examples.

[Preparation of Sphingolipid]

The sphingolipid used in the following Experimental Examples 1 to 3 was readied and prepared as follows.

As animal ceramide (Cer), C6 ceramide (d18:1/6:0, N-hexanoyl-D-erythro-sphingosine), C18 ceramide (d18:1/18:0, N-stearoyl-D-erythro-sphingosine), and C24 ceramide (d18:1/24:0, N-lignocelloyl-D-erythro-sphingosine) were used, and as sphingomyelin (SM), C6 sphingomyelin (d18:1/6:0, N-hexanoyl-D-erythro-sphingosylphosphorylcholine), C18 sphingomyelin (d18:1/18:0, N-stearoyl-D-erythro-sphingosylphosphorylcholine), and C24 sphingomyelin (d18:1/24:0, N-lignoceroyl-D-erythro-sphingosylphosphorylcholine) manufactured by Avanti Polar Lipids Inc., were used. These animal ceramides are all purified lipids manufactured by Avanti Polar Lipids Inc.

As the sphingomyelin (SM), C6 sphingomyelin (d18:1/6:0, N-hexanoyl-D-erythro-sphingosylphosphorylcholine), C18 sphingomyelin (d18:1/18:0, N-stearoyl-D-erythro-sphingosylphosphorylcholine), and C24 sphingomyelin (d18:1/24:0, N-lignoceroyl-D-erythro-sphingosylphosphorylcholine) manufactured by Avanti Polar Lipids Inc., were used. All of these sphingomyelins are purified lipids manufactured by Avanti Polar Lipids Inc.

C18 glucosylceramide (d18:1/18:0, D-glucosyl-β-1,1′-N-stearoyl-D-erythro-sphingosine) was used as glucosylceramide. The glucosylceramide is purified lipids manufactured by Avanti Polar Lipids Inc.

As konjac-derived glucosylceramide (kGluCer), purified lipid (NS170302 glucosylceramide, from Konjac, purity ≥99% (TLC)) was used. The konjac-derived glucosylceramide is manufactured by Nagara Science Co., Ltd.

As the konjac-derived ceramide (kCer), those obtained by hydrolyzing the konjac-derived glucosylceramide using Rhodococcus erythropolis-derived endoglycosidase (EGCase I), liberating glucose, and purifying the konjac-derived glucosylceramide were used.

Note that in the notation “dw:x/y:z” regarding the sphingolipid, ‘d’ indicates that the number of hydroxyl groups in the sphingoid moiety of ceramide per molecule is 2, “w:x” indicates the carbon atom w of the sphingoid moiety of ceramide per molecule and the number x of unsaturated bonds, and “y:z” indicates the carbon atom y of the fatty acid moiety and the number z of unsaturated bonds.

### Experimental Example 1

1. Experimental Method

(1) Treatment of Sphingolipid on Cultured Cell and Recovery of Exosome from Culture Supernatant

Human neuroblastoma-derived SH-SY5Y cell was used as the cultured cell. After the cell was inoculated in a 6-well plate at 2.5×10⁵ cells/well together with a medium (50% Ham's F12/50% E-MEM) and cultured at 37° C. for 24 hours, the animal ceramide (Cer), sphingomyelin (SM), or glucosylceramide (GuCer) sphingolipid was each added to each well. The sphingolipid was suspended in a serum-free medium (50% Ham's F12/50% E-MEM) containing 3% bovine serum albumin (BSA), and added so that the concentration in the well was 10 μM. The control was performed when the added amount of sphingolipid was 0 μM.

After 24 hours of the sphingolipid added, the culture supernatant (2 mL/well) was recovered, and the exosome was recovered from the culture supernatant. The centrifugation method was used to recover the exosome. Specifically, the recovered culture supernatant was sequentially subjected to centrifugation at 2,000 g for 10 minutes, 10,000 g for 30 minutes, and 100,000 g for 70 minutes to recover the exosome as a sediment.

(2) Measurement of Number of Exosome Particles

The exosome recovered by the centrifugation method was suspended in a HEPES/KCl buffer, and then the number of particles was measured with a nanoparticle analyzer qNano (Izon Inc.). The measured value was expressed as the number of particles per protein mass (mg) of the derived cell calculated by the BCA method. The number of particles is an average value of values obtained by performing measurement six times. In addition, the obtained values were subjected to a significant difference test (***P<0.001, **P<0.01, *P<0.05) by a one-way ANOVA method.

2. Experimental Result

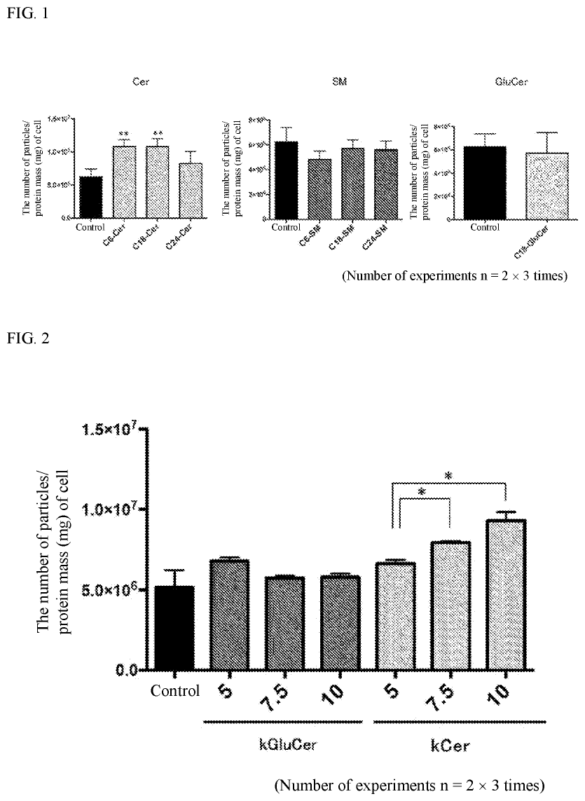

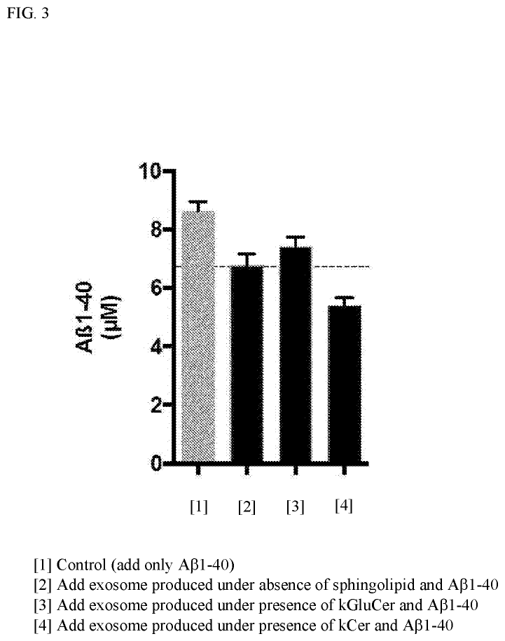

The results are shown in Table 1. From FIG. 1, it was recognized that the number of exosome particles in the neuronal cell line is significantly increased compared to the control when ceramide is added as compared to a case where sphingomyelin or glucosylceramide is added, and it was confirmed that the ceramide can promote the production of the ceramide. In addition, it was confirmed that the production of the exosome was further promoted when C6 ceramide and C18 ceramide were added among the ceramides.

### Experimental Example 2

1. Experimental Method

Experimental Example 2 was performed under the same conditions as in the above Experimental Example 1 except that the konjac-derived glucosylceramide (kGluCer) or the konjac-derived ceramide (kCer) are used as the sphingolipid and sphingolipids having concentrations of 5 μM, 7.5 μM, or 10 μM, respectively, were added to the cell in place of a concentration of 0 μM (control), and the number of exosome particles was measured.

2. Experimental Result

The results are shown in Table 2. From FIG. 2, it was not recognized that when 5 to 10 μM of konjac-derived glucosylceramide (kGluCer) was added, the number of exosome particles was increased compared to the control, but it was recognized that when 5 to 10 μM of the konjac-derived ceramide (kCer) was added, the number of exosome particles was increased for the control. In addition, it was recognized that the number of exosome particles is significantly increased according to the added amount of konjac-derived ceramide. From these results, it was confirmed that the konjac-derived ceramide can promote the production of the exosome. In addition, it was confirmed that the number of exosome particles increased with the increase in the concentration of ceramide.

### Experimental Example 3

1. Experimental Method

(1) Treatment of Sphingolipid on Cultured Cell and Recovery of Exosome from Culture Supernatant

Human neuroblastoma-derived SH-SY5Y cell was used as the cultured cell. After the cell was inoculated in a 6-well plate at 2.5×10⁵ cells/well together with a medium (50% Ham's F12/50% E-MEM) and cultured at 37° C. for 24 hours, the sphingolipid of the konjac-derived glucosylceramide (kGluCer) or the konjac-derived ceramide (kCer) was each added to each well. The sphingolipid was suspended in a serum-free medium (50% Ham's F12/50% E-MEM) containing 3% bovine serum albumin (BSA), and added so that the added amount was 10 μM. The same operation was performed even when the added amount of sphingolipid was 0 μM.

After 24 hours of the sphingolipid added, the culture supernatant (2 mL/well) was recovered, and the exosome was recovered from the culture supernatant. The centrifugation method was used to recover the exosome. Specifically, the recovered culture supernatant was sequentially subjected to centrifugation at 2,000 g for 10 minutes, 10,000 g for 30 minutes, and 100,000 g for 70 minutes to recover the exosome as a sediment.

(2) Addition of SH-SY5Y Cell-Derived Exosome and Amyloid β Protein to Microglia Cell, and Measurement of Concentration of Amyloid β Protein in Culture Medium

A mouse microglia cell line BV-2 cell was used as a cultured cell. After the cell is inoculated in a 6-well plate to be 2.5×10⁵ cells/well together with a medium (RPMI 1640) and cultured at 37° C. for 24 hours, the total amount of SH-SY5Y cell-derived exosome obtained above (total amount of exosome recovered from 2 mL of culture supernatant) and the amount of amyloid β protein (Aβ1-40) having a final concentration of 10 μM were added to each well. Note that the SH-SY5Y cell-derived exosome and the amyloid β protein were added after being suspended or dissolved in a serum-free medium (RPMI 1640). In addition, as the control, the same operation was performed even when only the amyloid β protein (Aβ1-40) was added without adding the SH-SY5Y cell-derived exosome.

The SH-SY5Y cell-derived exosome and the amyloid β protein were added, the culture supernatant was collected after 24 hours, and the concentration of amyloid β protein (Aβ1-40) in the culture supernatant was measured by an ELISA method.

2. Experimental Result

The results are shown in Table 3. The concentration of amyloid β protein was reduced by about 20% when the exosome produced under the presence of the konjac-derived ceramide is added as compared to a case where the exosome produced under the absence of the sphingolipid is added. In other words, it was confirmed from this result that even the exosome whose production was enhanced by the konjac-derived ceramide has, the binding ability with the amyloid β protein and the reactivity to the microglial cell, and the resolution of the amyloid β protein is improved with the increase in the production of the exosome.

1. An agent for promoting exosome production comprising a ceramide as an active ingredient.

2. The agent for promoting exosome production according to claim 1, wherein fatty acid constituting the ceramide has 6 to 26 carbon atoms.

3. The agent for promoting exosome production according to claim 1, wherein a sphingoid moiety constituting the ceramide has 18 carbon atoms.

4. The agent for promoting exosome production according to claim 1, wherein the agent for promoting exosome production is a food and drink for promoting a production of an exosome.

5. The agent for promoting exosome production according to claim 1, wherein the agent for promoting exosome production is a pharmaceutical product for promoting a production of an exosome.

6. The agent for promoting exosome production according to claim 1, wherein the agent for promoting exosome production is an Alzheimer's disease preventive agent.

7. A method for promoting a production of exosomes, comprising administering or ingesting an effective amount of ceramide to a person who needs to promote the production of the exosome.

8. The method for promoting a production of an exosome according to claim 7, wherein fatty acid constituting the ceramide has 6 to 26 carbon atoms.

9. The method for promoting a production of an exosome according to claim 7, wherein a sphingoid moiety constituting the ceramide has 18 carbon atoms.

10. The method for promoting a production of an exosome according to claim 7, wherein a food or drink containing the ceramide is ingested.

11. The method for promoting a production of an exosome according to claim 7, wherein a pharmaceutical product containing the ceramide is administrated.

12. The method for promoting a production of an exosome according to claim 7, wherein the person who needs to promote the production of the exosome is a person who needs to prevent Alzheimer's disease, and is administrated with or ingests the effective amount of the ceramide for prevention of the Alzheimer's disease.

13. The method for promoting a production of an exosome according to claim 12, wherein the person who needs to prevent the Alzheimer's disease is a person who is concerned about an onset of familial Alzheimer's disease.
